(19) United States
(12) Patent Application Publication
(10) Pub. No.: US 2020/0196848 A1
(43) Pub. Date: Jun. 25, 2020

(54) Lighting Module of Medical Endoscope and the Method of Making the Same

(71) Applicant: LIMIT OPTICS CO., LTD., Taichung (TW)

(72) Inventor: RUNG DE LIN, Taichung (TW)

(73) Assignee: Limit Optics Co., Ltd.

(21) Appl. No.: 16/229,592

(22) Filed: Dec. 21, 2018

(51) Int. Cl. A61B 1/06; G02B 23/24; A61B 1/00; A61B 1/05

(52) U.S. Cl. CPC A61B 1/0684; A61B 1/0676; A61B 1/00163; A61B 1/00096; A61B 1/05; G02B 23/2461

(57) ABSTRACT

A medical endoscope includes a circuit board, on which an image capture device and a LED bare die are mounted. The LED die is attached to the circuit board and electrically connected to a conductor pattern of the circuit board by conductive members. Next, a sealing member is coated onto the circuit board to embed the LED bare and the conducting members therein. The medical endoscope will have smaller size than the prior arts.

### BACKGROUND OF THE INVENTION

### 1. Technical Field

The present invention relates to medical equipment, and more particularly to a lighting module of a medical endoscope and the method of making the same.

### 2. Description of Related Art

Endoscopy is a common medical procedure in the hospitals to examine the interior of a hollow organ or cavity of the body. Endoscopy may be used for many medical purposes, such as investigation of symptoms, confirmation of a diagnosis, and giving treatment. The common endoscopes include cystoscopy, esophagogastroduodenoscopy, colonoscopy, bronchoscope, and celioscope.

Typically, the endoscopy procedure inserts a thin and long endoscope into human body. However, it will make the patient uncomfortable. The thinner endoscope may reduce the uncomfortable feeling, and in some circumstances, the patient will be anesthetized before endoscopy procedure.

A conventional endoscope includes an image capturing device and a lighting device. The common image capturing device is micro CMOS, and the common lighting device is LED. The LED is attached on a circuit by surface-mount technology (SMT).

However, the conventional endoscope has a limitation in size. It can't reduce the size for the modern design.

### BRIEF SUMMARY OF THE INVENTION

In view of the above, the primary objective of the present invention is to provide a medical endoscope and the method of making the same, which may sufficiently reduce its size.

In order to achieve the objective of the present invention, a medical endoscope includes a substrate provided with a conductor pattern; an image capturing device provided on the substrate and electrically connected to the conductor pattern; at least a LED bare die directly attached to the substrate and electrically connected to the conductor pattern by conductive members; and at least a sealing member provided to the substrate to embed the LED bare die and the conductive members.

In an embodiment, a glue is provided between the LED bare die and the substrate to attach the LED bare die to the substrate.

In an embodiment, the conductor pattern of the substrate has a plurality of bonding pads, to which ends of the conductive member are connected.

In an embodiment, the sealing member is transparent and has a predetermined color.

The present invention further provides a method of mounting a LED bare die onto a substrate of a medical endoscope, including the following steps:

A. Attach a LED bare die on a predetermined position of a substrate;

B. Electrically connect the LED bare die to a conductor pattern of the substrate by a plurality of conductive members;

C. Coat a sealing member onto the substrate and embedding the LED bare die and the conductive members therein; and

D. Cure the sealing member.

In an embodiment, the method further includes the step of cleaning the substrate and eliminating static electricity of the substrate before the step A.

In an embodiment, the step A includes the following steps:

Provide a glue on the predetermined position of the substrate;

Press the LED bare die onto the glue; and

Curing the glue.

In an embodiment, the method further includes testing an electrical connection between the LED bare die and the conductor pattern of the substrate after the step B and after the step D.

In an embodiment, the conductor pattern of the substrate has a plurality of bonding pads, the conductive members have ends connected to the bonding pads in the step B, and the sealing member embeds at least a portion of each of the bonding pads in the step C.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1 is an exploded view of a first preferred embodiment of the present invention;

FIG. 2 is a front view of the first preferred embodiment of the present invention;

FIG. 3 is a flow chart of the first preferred embodiment of the present invention;

FIG. 4 is a front view of a second preferred embodiment of the present invention; and

FIG. 5 is a front view of a third preferred embodiment of the present invention.

### DETAILED DESCRIPTION OF THE INVENTION

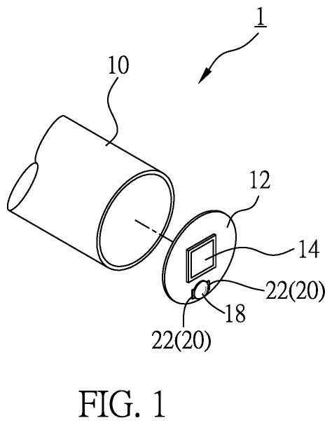

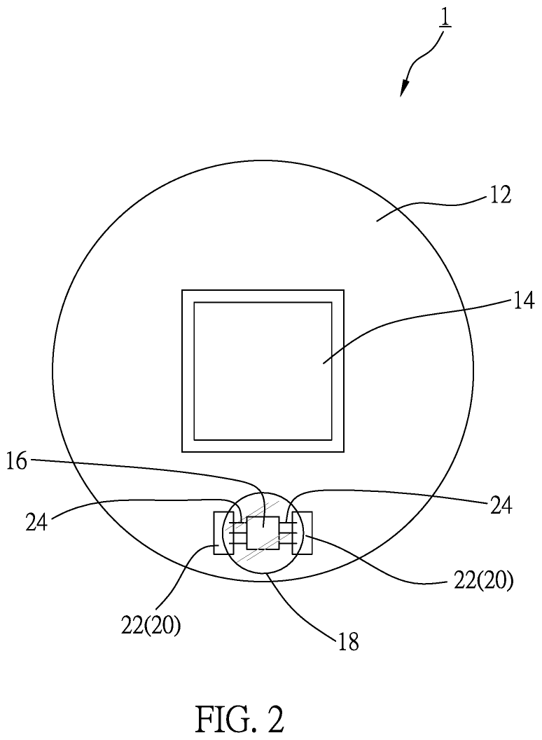

FIG. 1 shows a medical endoscope 1 of the first preferred embodiment of the present invention, including a housing 10, in which a substrate 12, an image capturing device 14, a LED bare die 16, and a sealing member 18, and the image capturing device 14, the LED bare die 16, and the sealing member 18 are provided on the substrate 12 (referring to FIG. 2). The substrate 12 has a conductor pattern 20, and the conductor pattern 20 as several bonding pads 22 to be electrically connected to the image capturing device 14 and the LED bare die 16.

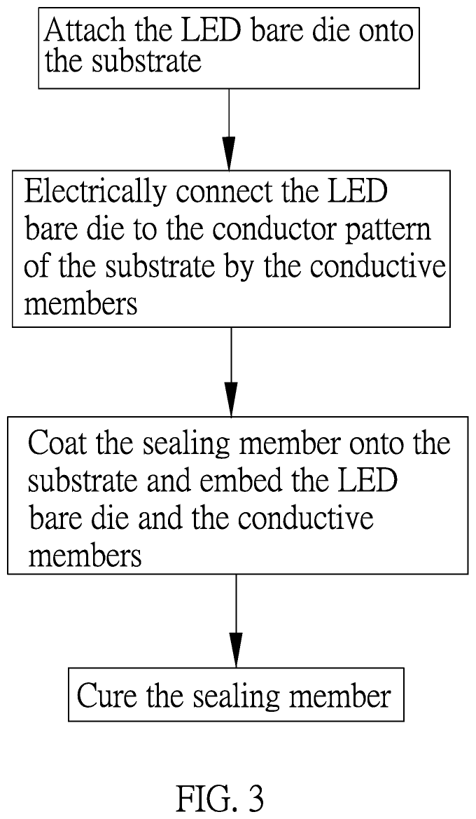

As shown in FIG. 3, a method of mounting the LED bare die 16 onto the substrate 12 includes the following steps:

A. Attach the LED bare die 16 on a predetermined position of the substrate 12.

In the first preferred embodiment, the substrate 12 is cleaned and processed for eliminating static electricity of the substrate 12. Next, provide a glue on the predetermined position of the substrate 12, and then press the LED bare die 16 onto the adhesive. After that, the substrate 12 is sent to an oven for curing the adhesive and for firmly attaching the LED bare die 16 on the substrate 12. The temperature and curing time are depended on the quantity and the content of the glue.

B. Electrically connect the LED bare die 16 to the conductor pattern 20 of the substrate 12 by conductive elements 24.

A process of electrically connecting the LED bare die 16 to the substrate 12 includes wire bonding, flip chip, and tape automatic bonding (TAB). In the first preferred embodiment, wire bonding process is used to electrically connect the LED bare die 16 to the substrate 12. The conductive elements 24 are conductive wires, each of which has opposite ends connected to a pad of the LED bare die 16 and one of the bonding pads 22 of the conductor pattern 20 of the substrate 12, respectively.

After the LED bare die 16 is electrically connected to the conductor pattern 20 of the substrate 12, a testing procedure is performed to check whether the LED bare die 16 is correctly connected to the conductor pattern 20 of the substrate 12. If it passes the testing procedure, it can continue the following steps, and if it does not, the LED bare die 16 and the substrate 12 may be repaired or abandoned.

C. Coat the sealing member 18 onto the substrate 12 to embed the LED bare die 16 and the conductive elements 24 therein.

In the first preferred embodiment, except the LED bare die 16 and the conductive elements 24, at least a portion of each of the bonding pads 22 of the conductor pattern 20 are embedded in the sealing member 18.

D. Cure the sealing member 18.

The substrate 12 is sent to an oven to heat the sealing member 18 under a predetermined temperature for a predetermined time. The temperature and curing time are depended on the quantity and the content of the sealing member 18. In the first preferred embodiment, the cured sealing member 18 is round in a top view. In addition, the cured sealing member 18 is transparent or has a predetermined color, such as blue and yellow, to pass a light provided by the LED bare die 16 and let the light have a predetermined color.

After curing, a testing procedure is performed is performed again to check whether the LED bare die 16 is correctly connected to the conductor pattern 20 of the substrate 12. If it passes the testing procedure, it can continue the following steps, and if it does not, the LED bare die 16 and the substrate 12 may be repaired or abandoned.

With the steps as described above, the LED bare die 16 is directly bonded onto the substrate 12 to reduce a sufficient size of the substrate 12. According to a test of the inventor, the substrate made by the conventional process has a diameter over 3 mm. On the contrary, a diameter of the substrate 12, which is made by the method of the present invention, is 2.6 mm. In conclusion, with the method of the present invention, it may reduce about 10% in diameter.

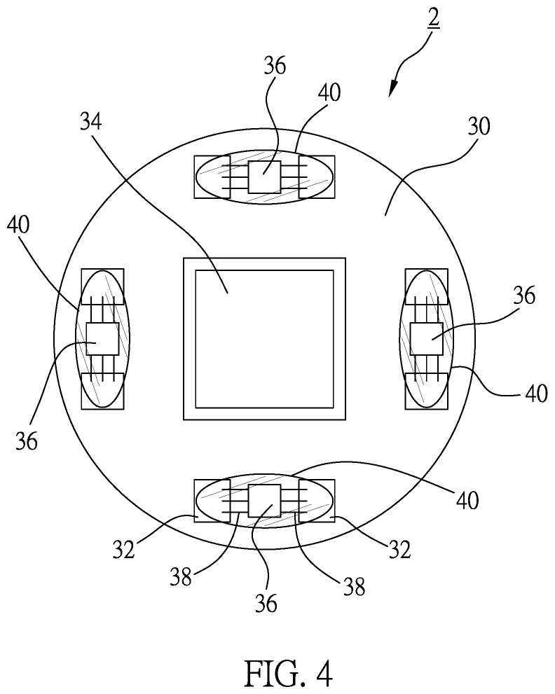

FIG. 4 shows a medical endoscope 2 of the second preferred embodiment of the present invention, which is similar to the first preferred embodiment, including a substrate 30 having a conductor pattern 32, an image capturing device 34 attached to the substrate 20 and electrically connected to the conductor pattern 32, four LED bare dies 36 attached to the substrate 20 and electrically connected to the conductor pattern 32 by a plurality of conductive members 38, and four sealing members 40 provided on the substrate 20 to embed the LED bare dies 36 and the conductive members 38 respectively. a method of making the medical endoscope 2 of the second preferred embodiment is the same as above. The cured sealing members 40 are elliptical in top views. According to the test by the inventor, a diameter of the substrate 30 of the second preferred embodiment is about 2.6 mm.

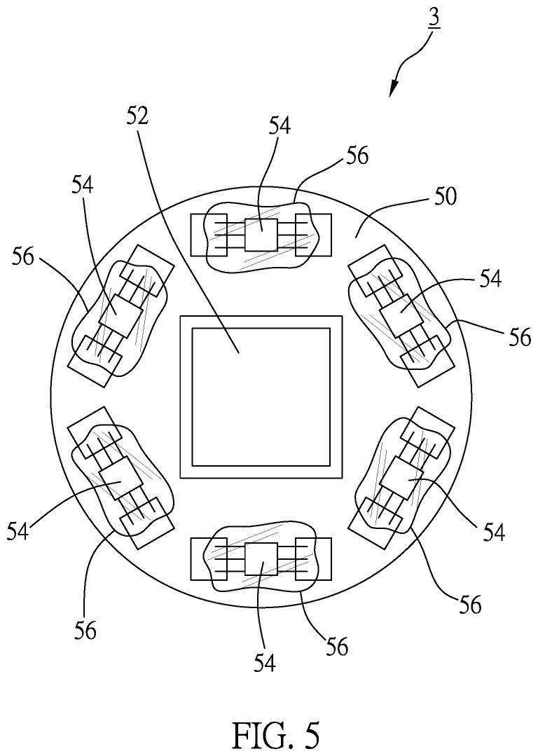

FIG. 5 shows a medical endoscope 3 of the third preferred embodiment of the present invention, which is similar to the first and the second preferred embodiments, including a substrate 50, an image capturing device 52, eight LED bare dies 54, and eight sealing members 56. The different parts are that a number of the LED bare dies 54 is eight, and the cured sealing members 56 are random in top views. According to the test by the inventor, a diameter of the substrate 30 of the second preferred embodiment is about 2.6 mm.

It must be pointed out that the embodiments described above are only some preferred embodiments of the present invention. All equivalent structures which employ the concepts disclosed in this specification and the appended claims should fall within the scope of the present invention.

What is claimed is:

1. A medical endoscope, comprising:
a substrate provided with a conductor pattern;
an image capturing device provided on the substrate and electrically connected to the conductor pattern;
at least a LED bare die directly attached to the substrate and electrically connected to the conductor pattern by conductive members; and
at least a sealing member provided to the substrate to embed the LED bare die and the conductive members.

2. The medical endoscope of claim 1, wherein a glue is provided between the LED bare die and the substrate to attach the LED bare die to the substrate.

3. The medical endoscope of claim 1, wherein the conductor pattern of the substrate has a plurality of bonding pads, to which ends of the conductive member are connected.

4. The medical endoscope of claim 1, wherein the sealing member is transparent and has a predetermined color.

5. A method of mounting a LED bare die onto a substrate of a medical endoscope, comprising the steps of:
A. attaching a LED bare die on a predetermined position of a substrate;
B. electrically connecting the LED bare die to a conductor pattern of the substrate by a plurality of conductive members;
C. coating a sealing member onto the substrate and embedding the LED bare die and the conductive members therein; and
D. curing the sealing member.

6. The method of claim 5, further comprising cleaning the substrate and eliminating static electricity of the substrate before the step A.

7. The workstation of claim 5, wherein the step A comprises:
providing a glue on the predetermined position of the substrate;
pressing the LED bare die onto the glue; and
curing the glue.

8. The workstation of claim 6, further comprising testing an electrical connection between the LED bare die and the conductor pattern of the substrate after the step B.

9. The workstation of claim 1, wherein the conductor pattern of the substrate has a plurality of bonding pads, the conductive members have ends connected to the bonding pads in the step B, and the sealing member embeds at least a portion of each of the bonding pads in the step C.

10. The workstation of claim 1, further comprising testing an electrical connection between the LED bare die and the conductor pattern of the substrate after the step D.
